(19) United States
(12) Patent Application Publication
(10) Pub. No.: US 2025/0107951 A1
(43) Pub. Date: Apr. 3, 2025

(54) Portable Bowel Cup and Liner Device

(71) Applicant: Anna Coleman, Jacksonville, FL (US)

(72) Inventor: Anna Coleman, Jacksonville, FL (US)

(21) Appl. No.: 18/522,441

(22) Filed: Nov. 29, 2023

Related U.S. Application Data
(60) Provisional application No. 63/586,142, filed on Sep. 28, 2023.

(51) Int. Cl. A61G 9/00; A61G 9/02

(52) U.S. Cl. CPC A61G 9/003; A61G 9/02

(57) ABSTRACT

A portable bowel cup and liner device is provided. The device is comprised of a body with at least one flared lip. The body receives at least one removable liner. The lip of the body can be placed around the buttocks and anus of a user lying on their side such that waste from the user is captured within the liner. Then, the liner can be removed from the body, closed via a fastener of the liner, and then disposed of. The body may be comprised of a weighted base to inhibit movement of the body.

### CROSS-REFERENCE TO RELATED APPLICATION

The present application claims priority to, and the benefit of, U.S. Provisional Application No. 63/586,142, which was filed on Sep. 28, 2023, and is incorporated herein by reference in its entirety.

### FIELD OF THE INVENTION

The present invention relates generally to the field of waste-collecting devices. More specifically, the present invention relates to a portable bowel cup and liner device comprised of a body with a flared lip that receives a liner. The device can be positioned at the buttocks of a user lying on their side such that waste from the user is collected in the liner, which can then be removed from the body, closed, and disposed of. Accordingly, the present disclosure makes specific reference thereto. Nonetheless, it is to be appreciated that aspects of the present invention are also equally applicable to other like applications, devices, and methods of manufacture.

### BACKGROUND

Individuals who find themselves restricted to a bed due to severe physical injuries, chronic conditions, or other medical complications will, as a natural course of life, need to address their bodily functions such as bowel movements. While there are tools like bedpans, commodes, and specialized medical units designed to aid these patients, they are not foolproof. The use of such devices can often lead to unintended spillage or leakage, resulting in soiled undergarments and stained bed linens. These incidents necessitate additional work for the medical staff, often involving more than one healthcare professional, to ensure that the patient and the surrounding environment are clean.

Furthermore, beyond the practical implications of such situations, there lies a deeper emotional and psychological impact on the patient. When these mishaps occur, it's not just about the physical mess, there's an undeniable erosion of the individual's dignity. Many patients already grapple with feelings of vulnerability due to their condition, and incidents where they lose control over basic bodily functions can exacerbate these sentiments. The simple act of having soiled clothes or linens can deeply affect a person's self-worth and confidence, making them feel more like a burden than a valued individual.

Additionally, even in situations where the bedpan or medical device has been used correctly and with the utmost care, disposing of the waste can still be an intricate process. Often, it's not just about emptying the contents but ensuring that the equipment is sanitized and ready for subsequent use. This process demands both time and attention to detail, putting additional strain on the healthcare system and its professionals. The ripple effect of these seemingly small incidents showcases the need for more innovative solutions and approaches to cater to bedridden patients' needs.

Therefore, there exists a long-felt need in the art for an improved waste-collecting device for bedridden users. There also exists a long-felt need in the art for a portable bowel cup and liner device that can be used to collect waste from bedridden users. More specifically, there exists a long-felt need in the art for a portable bowel cup and liner device that can be used to collect waste from bedridden users wherein the device minimizes the possibility of spillage or leakage of waste. Further, there exists a long-felt need in the art for a portable bowel cup and liner device that can be used to collect waste from bedridden users wherein the device allows a user to easily dispose of the collected waste.

The subject matter disclosed and claimed herein, in one embodiment thereof, comprises a portable bowel cup and liner device. The device is comprised of a body with at least one flared lip. The body receives at least one removable liner. The lip of the body can be placed around the buttocks and anus of a user lying on their side such that waste from the user is captured within the liner. Then, the liner can be removed from the body, closed via a fastener of the liner, and then disposed of. The body may be comprised of a weighted base to inhibit movement of the body.

In this manner, the portable bowel cup and liner device of the present invention accomplishes all the foregoing objectives and provides an improved waste-collecting device for bedridden users. More specifically, the device can be used to collect waste from bedridden users wherein the device minimizes the possibility of spillage or leakage of waste via the design of the body and lip. Further, the device allows a user to easily dispose of the collected waste via the liner.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a portable bowel cup and liner device. A device is provided comprised of an interior space that receives at least one disposable liner. The body can be positioned around/near the buttocks of a user in order to collect waste from the user within the liner. Then, the liner can be removed from the body and disposed of.

The body is preferably generally rectangular and is formed by two sets of generally parallel side walls and a bottom wall. In one embodiment, at least one wall may be comprised of at least one handle (of any type) to allow the body to be easily positioned and handled. The body is also comprised of at least one flared lip that surrounds the opening of the body. The lip preferably makes contact around the buttocks and/or anus of the user. The shape of the flared lip ensures maximum capturing of waste and prevents spillage.

The liner is preferably waterproof and odor-proof to prevent waste leakage and smell. During use, the liner can be placed inside the interior space of the body and secured around the lip of the opening. The liner is comprised of at least one opening that captures waste from the user inside the liner. The liner is preferably comprised of at least one fastener that allows the liner to be closed into a bag-like shape once waste has been collected within the liner. In one embodiment, the liner is comprised of at least one cleaning item stored within the liner. The item may be in the form of a sealed, sterile tissue or moistened wipe.

The present invention is also comprised of a method of using the device. First, a device is provided comprised of a body with an interior space and at least one flared lip and a liner received by the interior space. Then, the liner can be placed into the interior space and secured around the lip (i.e., folded over the lip). Then, the body can be placed into at least one base. Next, the body and base can be placed horizontally around the buttocks of a user lying on their side, such that the buttocks contacts and/or is received by the lip. Once the user's waste is collected by the liner, the liner can be formed into a bag-like shape via at least one fastener of the liner. Then, the liner can be disposed of.

The present invention is also comprised of a second method of using the device. First, a device is provided comprised of a body with an interior space and at least one flared lip and a liner received by the interior space, and at least one cleaning item attached to the liner. Then, the liner can be placed into the interior space and secured around the lip (i.e., folded over the lip) after the cleaning item is removed from the liner. Then, the body can be placed into at least one base. Next, the body and base can be placed horizontally around the buttocks of a user lying on their side, such that the buttocks contacts and/or is received by the lip. Once the user's waste is collected by the liner, the liner can be formed into a bag-like shape via at least one fastener of the liner. Then, the liner can be disposed of. Finally, the cleaning item can be used to clean the buttocks/anus of the user.

Accordingly, the portable bowel cup and liner device of the present invention is particularly advantageous as it provides an improved waste-collecting device for bedridden users. More specifically, the device can be used to collect waste from bedridden users wherein the device minimizes the possibility of spillage or leakage of waste via the design of the body and lip. Further, the device allows a user to easily dispose of the collected waste via the liner. In this manner, the portable bowel cup and liner device overcomes the limitations of existing bed pans and bedridden waste collection devices known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates an exploded view of a body and a base of one potential embodiment of a portable bowel cup and liner device of the present invention in accordance with the disclosed architecture;

FIG. 2 illustrates a perspective view of a body and a base of one potential embodiment of a portable bowel cup and liner device of the present invention in accordance with the disclosed architecture;

FIG. 3 illustrates a perspective view of a liner, body, and a base of one potential embodiment of a portable bowel cup and liner device of the present invention in accordance with the disclosed architecture;

FIG. 4 illustrates a perspective view of a liner of one potential embodiment of a portable bowel cup and liner device of the present invention in accordance with the disclosed architecture;

FIG. 5 illustrates a perspective view of a body, a liner, and a base of one potential embodiment of a portable bowel cup and liner device of the present invention while placed around the buttocks of a user in accordance with the disclosed architecture;

FIG. 6 illustrates a flowchart of a method of using one potential embodiment of a portable bowel cup and liner device of the present invention in accordance with the disclosed architecture; and

FIG. 7 illustrates a flowchart of a method of using one potential embodiment of a portable bowel cup and liner device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved waste-collecting device for bedridden users. There also exists a long-felt need in the art for a portable bowel cup and liner device that can be used to collect waste from bedridden users. More specifically, there exists a long-felt need in the art for a portable bowel cup and liner device that can be used to collect waste from bedridden users wherein the device minimizes the possibility of spillage or leakage of waste. Further, there exists a long-felt need in the art for a portable bowel cup and liner device that can be used to collect waste from bedridden users wherein the device allows a user to easily dispose of the collected waste.

The present invention, in one exemplary embodiment, is comprised of a portable bowel cup and liner device comprised of an interior space that receives at least one disposable liner. The body can be positioned around/near the buttocks of a user in order to collect waste from the user within the liner. Then, the liner can be removed from the body and disposed of.

The body is preferably generally rectangular and is formed by two sets of generally parallel side walls and a bottom wall. In one embodiment, at least one wall may be comprised of at least one handle (of any type) to allow the body to be easily positioned and handled. The body is also comprised of at least one flared lip that surrounds the opening of the body that makes contact around the buttocks and/or anus of the user. The shape of the flared lip ensures maximum capturing of waste and prevents spillage.

The liner is preferably waterproof and odor-proof to prevent waste leakage and smell. During use, the liner can be placed inside the interior space of the body and secured around the lip of the opening. The liner is comprised of at least one opening that captures waste from the user inside the liner. The liner is preferably comprised of at least one fastener that allows the liner to be closed into a bag-like shape once waste has been collected within the liner. In one embodiment, the liner is comprised of at least one cleaning item stored within the liner. The item may be in the form of a sealed, sterile tissue or moistened wipe.

The present invention is also comprised of a method of using the device. First, a device is provided comprised of a body with an interior space and at least one flared lip and a liner received by the interior space. Then, the liner can be placed into the interior space and secured around the lip (i.e., folded over the lip). Then, the body can be placed into at least one base. Next, the body and base can be placed horizontally around the buttocks of a user lying on their side, such that the buttocks contacts and/or is received by the lip. Once the user's waste is collected by the liner, the liner can be formed into a bag-like shape via at least one fastener of the liner. Then, the liner can be disposed of.

The present invention is also comprised of a second method of using the device. First, a device is provided comprised of a body with an interior space and at least one flared lip and a liner received by the interior space, and at least one cleaning item attached to the liner. Then, the liner can be placed into the interior space and secured around the lip (i.e., folded over the lip) after the cleaning item is removed from the liner. Then, the body can be placed into at least one base. Next, the body and base can be placed horizontally around the buttocks of a user lying on their side, such that the buttocks contacts and/or is received by the lip. Once the user's waste is collected by the liner, the liner can be formed into a bag-like shape via at least one fastener of the liner. Then, the liner can be disposed of. Finally, the cleaning item can be used to clean the buttocks/anus of the user.

Accordingly, the portable bowel cup and liner device of the present invention is particularly advantageous as it provides an improved waste-collecting device for bedridden users. More specifically, the device can be used to collect waste from bedridden users wherein the device minimizes the possibility of spillage or leakage of waste via the design of the body and lip. Further, the device allows a user to easily dispose of the collected waste via the liner. In this manner, the portable bowel cup and liner device overcomes the limitations of existing bed pans and bedridden waste collection devices known in the art.

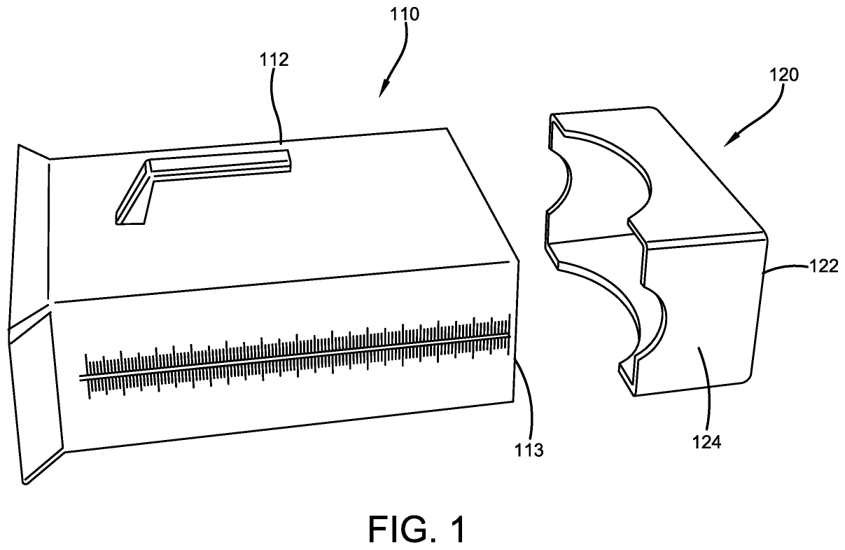

Referring initially to the drawings, FIG. 1 illustrates an exploded view of a body 110 and a base 120 of one potential embodiment of a portable bowel cup and liner device 100 of the present invention in accordance with the disclosed architecture. A device 100 is provided comprised of an interior space 111 that receives at least one disposable liner 130. The body 110 can be positioned around/near the buttocks 12 of a user 10 in order to collect waste from the user within the liner 130. Then, the liner 130 can be removed from the body 110 and disposed of.

The body 110 is preferably made of a plastic material but may be made from any material. In one embodiment, the body 110 is transparent to allow a medical professional or other user to view the amount of waste collected within the device 100. In this embodiment, the body 110 may be comprised of at least one measuring line 140 for measuring the quantity of waste collected. The line 140 may be any increment. The line 140 may also be found on the liner 130.

The body 110 is preferably generally rectangular but may be any shape. The body 110 is preferably formed by two sets of generally parallel side walls 112 and a bottom wall 113. In one embodiment, at least one wall 112 may be comprised of at least one handle 118 (of any type) to allow the body 110 to be easily positioned and handled.

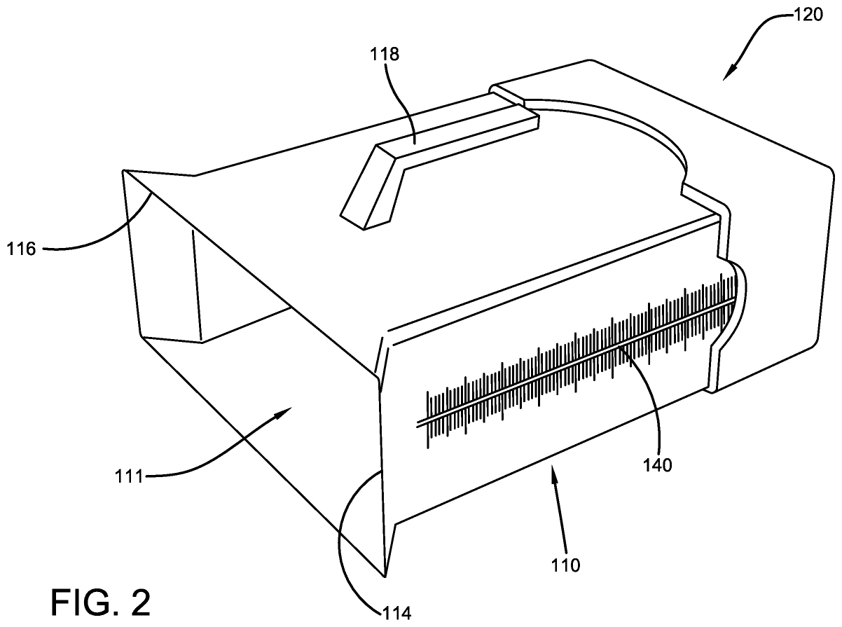

The body 110 may be received by at least one base 120 in one embodiment, as also seen in FIG. 2. The base 120 may be comprised of two sets of generally parallel side walls 124 and a bottom wall 122 that receive the side walls 112 and bottom wall 113 of the body 110. The base 120 may be weighted in order to prevent the body 110 from moving, rolling, or shifting position while being used.

The body 110 is also comprised of at least one flared lip 116. The lip 116 surrounds the opening 114 of the body 110. The lip 116 preferably makes contact around the buttocks 12 and/or anus of the user 10. The shape of the flared lip 116 ensures maximum capturing of waste and prevents spillage.

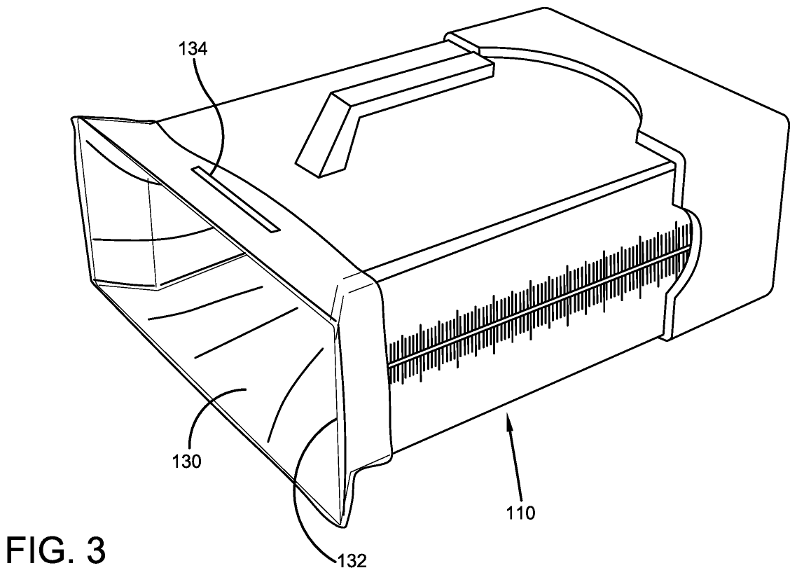

The liner 130 may be made from any type of disposable, medical-grade material known in the art. The liner 130 is preferably waterproof and odor-proof to prevent waste leakage and smell. In one embodiment, the exterior of the liner 130 may be scented. During use, the liner 130 can be placed inside the interior space 111 of the body 110 and secured around the lip 116 of the opening 114, as seen in FIG. 3. The liner 130 is comprised of at least one opening 132 that captures waste from the user 10 inside the liner 130.

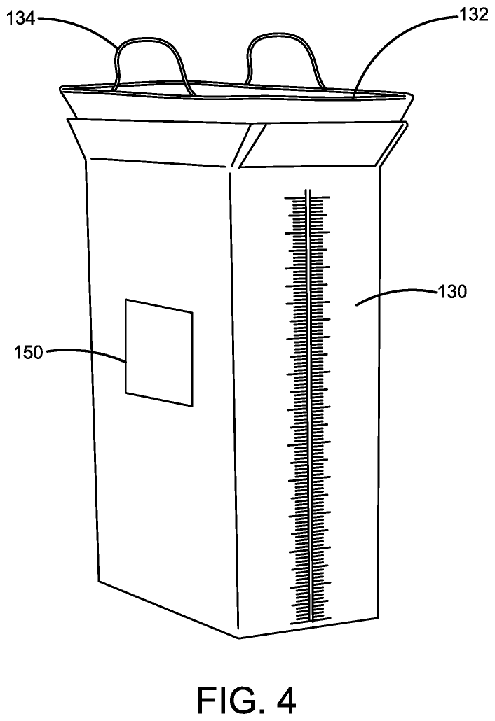

The liner 130 is preferably comprised of at least one fastener 134, as seen in FIG. 4. The fastener 134 is preferably a drawstring fastener. The fastener 134 allows the liner 130 to be closed into a bag-like shape once waste has been collected within the liner 130. Then, the liner 130 can be removed from the body 110 and disposed of. In different embodiments, the fastener 134 may be other fastener types such as, but not limited to, adhesive.

In one embodiment, the liner 130 is comprised of at least one cleaning item 150 stored within the liner 130. The item 150 may be in the form of a sealed, sterile tissue or moistened wipe. The item 150 can be removed prior to positioning the body 110 and liner 130 for waste collection. In this manner, the item 150 can then be used to clean the buttocks 12 of the user 10 after defecation.

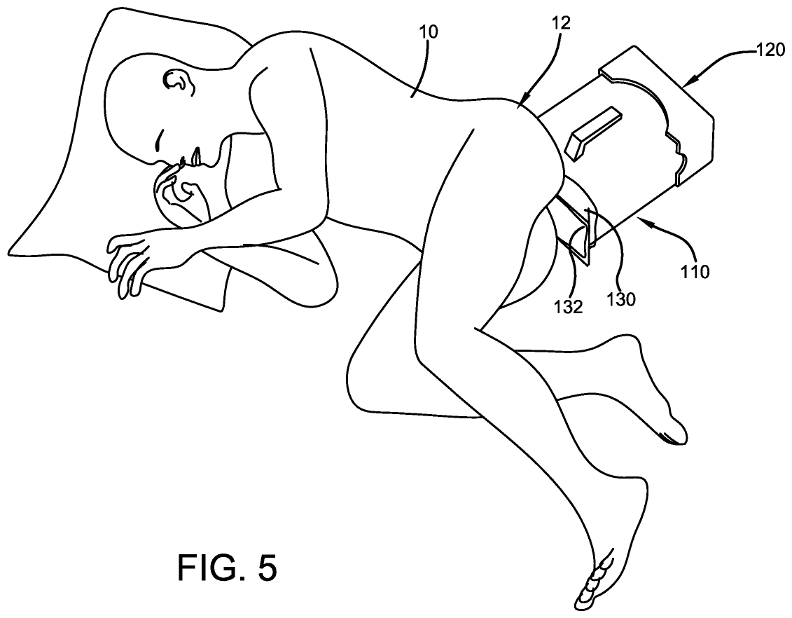

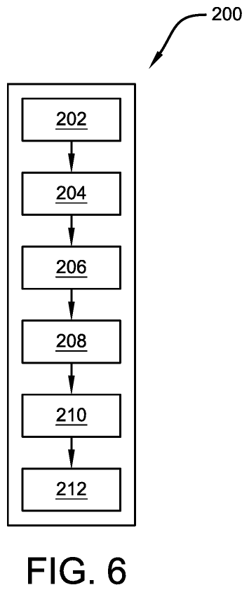

The present invention is also comprised of a method of using 200 the device 100, as seen in FIG. 6. First, a device 100 is provided comprised of a body 110 with an interior space 111 and at least one flared lip 116 and a liner 130 received by the interior space 111 [Step 202]. Then, the liner 130 can be placed into the interior space 111 and secured around the lip 116 (i.e., folded over the lip 116) [Step 204]. Then, the body 110 can be placed into at least one base 120 [Step 206]. Next, the body 110 and base 120 can be placed horizontally around the buttocks 12 of a user 10 lying on their side, such that the buttocks 12 contacts and/or is received by the lip 116, as seen in FIG. 5 [Step 208]. Once the user's 10 waste is collected by the liner 130, the liner 130 can be formed into a bag-like shape via at least one fastener 134 of the liner 130 [Step 210]. Then, the liner 130 can be disposed of [Step 212].

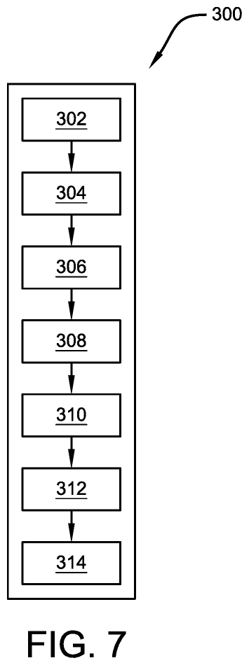

The present invention is also comprised of a method of using 300 the device 100, as seen in FIG. 7. First, a device 100 is provided comprised of a body 110 with an interior space 111 and at least one flared lip 116 and a liner 130 received by the interior space 111, and at least one cleaning item 150 attached to the liner 130 [Step 302]. Then, the liner 130 can be placed into the interior space 111 and secured around the lip 116 (i.e., folded over the lip 116) after the cleaning item 150 is removed from the liner 130 [Step 304]. Then, the body 110 can be placed into at least one base 120 [Step 306]. Next, the body 110 and base 120 can be placed horizontally around the buttocks 12 of a user 10 lying on their side, such that the buttocks 12 contacts and/or is received by the lip 116, as seen by FIG. 5 [Step 308]. Once the user's 10 waste is collected by the liner 130, the liner 130 can be formed into a bag-like shape via at least one fastener 134 of the liner 130 [Step 310]. Then, the liner 130 can be disposed of [Step 312]. Finally, the cleaning item 150 can be used to clean the buttocks/anus of the user 10 [Step 314].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “portable bowel cup and liner device” and “device” are interchangeable and refer to the portable bowel cup and liner device 100 of the present invention.

Notwithstanding the foregoing, the portable bowel cup and liner device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the portable bowel cup and liner device 100 as shown in the FIGS. are for illustrative purposes only, and that many other sizes and shapes of the portable bowel cup and liner device 100 are well within the scope of the present disclosure. Although the dimensions of the portable bowel cup and liner device 100 are important design parameters for user convenience, the portable bowel cup and liner device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A portable bowel cup and liner device comprising:
a body comprised of an interior space and a flared lip; and
a liner comprised of an opening and a fastener.

2. The portable bowel cup and liner device of claim 1, wherein the body is comprised of a handle.

3. The portable bowel cup and liner device of claim 1, wherein the liner is comprised of a waterproof material.

4. The portable bowel cup and liner device of claim 1, wherein the liner is comprised of an odor-proof material.

5. The portable bowel cup and liner device of claim 1, wherein the fastener is comprised of a drawstring fastener.

6. The portable bowel cup and liner device of claim 1, wherein the fastener is comprised of an adhesive fastener.

7. The portable bowel cup and liner device of claim 1, wherein the body is transparent.

8. The portable bowel cup and liner device of claim 7, wherein the body is comprised of a measuring line.

9. A portable bowel cup and liner device comprising:
a body comprised of an interior space and a flared lip;
a liner comprised of an opening and a fastener; and
a base that receives the body.

10. The portable bowel cup and liner device of claim 9, wherein the body is comprised of a handle.

11. The portable bowel cup and liner device of claim 9, wherein the liner is comprised of a waterproof material.

12. The portable bowel cup and liner device of claim 9, wherein the liner is comprised of an odor-proof material.

13. The portable bowel cup and liner device of claim 9, wherein the fastener is comprised of a drawstring fastener.

14. The portable bowel cup and liner device of claim 9, wherein the fastener is comprised of an adhesive fastener.

15. The portable bowel cup and liner device of claim 9, wherein the liner is comprised of a removable cleaning item.

16. The portable bowel cup and liner device of claim 15, wherein the removable cleaning item is comprised of a sterile tissue.

17. The portable bowel cup and liner device of claim 15, wherein the removable cleaning item is comprised of a moistened wipe.

18. A method of using a portable bowel cup and liner device, the method comprising the following steps:
providing a portable bowel cup and liner device comprised of a body comprised of an interior space and a flared lip, and a liner received by the interior space;
placing the liner in the interior space;
placing the body horizontally around a buttocks of a user lying on their side such that the buttocks are received by the lip;
forming the liner into a bag-like shape via a fastener of the liner once a waste from the user is collected by the liner; and
disposing of the liner.

19. The method of using a portable bowel cup and liner device of claim 18, wherein the fastener is comprised of a draw-string fastener.

20. The method of using a portable bowel cup and liner device of claim 18, wherein the body is comprised of a weighted base.
